(19) United States
(12) Patent Application Publication
(10) Pub. No.: US 2024/0257909 A1
(43) Pub. Date: Aug. 1, 2024

(54) BIOMARKERS FOR MITOCHONDRIAL AND METABOLIC DISORDERS

(71) Applicant: BOARD OF TRUSTEES OF THE UNIVERSITY OF ARKANSAS, Little Rock, AR (US)

(72) Inventors: Shilpa Iyer, Springdale, AR (US); Raj R. Rao, Springdale, AR (US); Shilpi Agrawal, Fayetteville, AR (US)

(73) Assignee: BOARD OF TRUSTEES OF THE UNIVERSITY OF ARKANSAS, Little Rock, AR (US)

(21) Appl. No.: 18/425,248

(22) Filed: Jan. 29, 2024

Related U.S. Application Data
(60) Provisional application No. 63/441,710, filed on Jan. 27, 2023.

(51) Int. Cl. G16B 25/10; G16B 20/20; G16B 40/20; G16H 50/20

(52) U.S. Cl. CPC G16B 25/10; G16B 20/20; G16B 40/20; G16H 50/20

(57) ABSTRACT

Embodiments of the present disclosure pertain to methods of assessing one or more mitochondrial or metabolic disorders in a subject by receiving one or more measured biomarker levels of the subject and correlating differentially expressed levels of the one or more measured biomarkers to one or more mitochondrial or metabolic disorders in the subject. Such methods may also include a step of making a treatment decision based on the assessment. Additional embodiments of the present disclosure pertain to a computing device for assessing one or more mitochondrial or metabolic disorders in a subject in accordance with the methods of the present disclosure.

### CROSS-REFERENCE TO RELATED APPLICATIONS

This application claims priority to U.S. Provisional Patent Application No. 63/441,710, filed on Jan. 27, 2023. The entirety of the aforementioned application is incorporated herein by reference.

### STATEMENT REGARDING FEDERALLY SPONSORED RESEARCH

This invention was made with government support under W81XWH-16-1-0181 awarded by the Department of Defense. The government has certain rights in the invention.

### BACKGROUND

Diagnosis of mitochondrial and metabolic disorders are challenging due to clinical variance. Numerous embodiments of the present disclosure aim to address the aforementioned challenges.

### SUMMARY

Embodiments of the present disclosure pertain to methods of assessing one or more mitochondrial or metabolic disorders in a subject. In some embodiments, the methods of the present disclosure include: receiving one or more measured biomarker levels of the subject; and correlating differentially expressed levels of the one or more measured biomarkers to one or more mitochondrial or metabolic disorders in the subject. In some embodiments, the one or more measured biomarker levels includes at least one of putrescine, N-acetyl putrescine, N-acetyl ornithine, N-acetyl spermidine, N-acetyl arginine, gamma amino butyric acid (GABA), opthalmic acid, S-adenosyl methionine, creatine, or combinations thereof.

In some embodiments, the methods of the present disclosure also include a step of making a treatment decision based on the assessment. In some embodiments, the treatment decision includes monitoring the course of the mitochondrial or metabolic disorder and/or administering a therapeutic agent to the subject. In some embodiments, the assessment may be repeated after implementing the treatment decision.

Additional embodiments of the present disclosure pertain to a computing device for assessing one or more mitochondrial or metabolic disorders in a subject. In some embodiments, the computing device includes one or more computer readable storage mediums having a program code embodied therewith. In some embodiments, the program code includes programming instructions for: receiving one or more measured biomarker levels of the subject; and correlating differentially expressed levels of the one or more measured biomarkers to one or more mitochondrial or metabolic disorders in the subject. In some embodiments, the program code further includes programming instructions for diagnosing the subject with one or more mitochondrial or metabolic disorders based on the correlation. In some embodiments, the program code further includes programming instructions for assessing the severity of one or more mitochondrial or metabolic disorders in the subject based on the correlation. In some embodiments, the program code further includes programming instructions for making a treatment decision based on the assessment.

### BRIEF DESCRIPTION OF THE DRAWINGS

A better understanding of the present invention can be obtained when the following detailed description is considered in conjunction with the following drawings, in which:

FIGS. 1A and 1B illustrate a method (FIG. 1A) and a computing device (FIG. 1B) for assessing one or more mitochondrial or metabolic disorders in a subject in accordance with various embodiments of the present disclosure.

FIGS. 2A-2C show volcano plots obtained from metabolomics analysis of SBG1 (FIG. 2A), SBG2 (FIG. 2B), and SBG3 (FIG. 2C) diseased fibroblast cell lines.

FIGS. 3A-3B show volcano plots obtained from metabolomics analysis of SBG4 (FIG. 3A) and SBG5 (FIG. 3B) diseased fibroblast cell lines.

FIGS. 4A-4B show volcano plots obtained from metabolomics analysis of SBG6 (FIG. 4A) and SBG7 (FIG. 4B) diseased fibroblast cell lines.

FIGS. 5A-5C show volcano plots obtained from metabolomics analysis of SBG8 (FIG. 5A), SBG9 (FIG. 5B), and SBG10 (FIG. 5B) diseased fibroblast cell lines.

FIG. 6 shows concentration analyses of different fibroblast cell lines based on metabolomic data, which indicate highly elevated levels of the metabolite “putrescine” in all ten diseased fibroblast cell lines when compared to control fibroblasts. ****-p<0.0001.

FIG. 7 shows concentration analyses of different fibroblast cell lines based on metabolomic data, which indicate highly elevated levels of the metabolite “N-acetyl putrescine” in all ten diseased fibroblast cell lines when compared to control fibroblasts. *-p<0.05; ***-p<0.001 ****-p<0.0001.

FIG. 8 shows concentration analyses of different fibroblast cell lines based on metabolomic data, which indicate highly elevated levels of the metabolite “N-acetyl ornithine” in nine out of the ten diseased fibroblast cell lines when compared to control fibroblasts. ns—not significant; **-p<0.01 ***-p<0.001 ****-p<0.0001.

FIG. 9 shows concentration analyses of different fibroblast cell lines based on metabolomic data, which indicate highly elevated levels of the metabolite “N-acetyl spermidine” in eight out of the ten diseased fibroblast cell lines when compared to control fibroblasts. ns—not significant; ***-p<0.001 ****-p<0.0001.

FIG. 10 shows concentration analyses of different fibroblast cell lines based on metabolomic data, which indicate highly elevated levels of the metabolite “N-acetyl Arginine” in all ten diseased fibroblast cell lines when compared to control fibroblasts. ***-p<0.001 ****-p<0.0001.

FIG. 11 shows concentration analyses of different fibroblast cell lines based on metabolomic data, which indicate highly elevated levels of the metabolite “GABA—Gamma Amino Butyric acid” in all ten diseased fibroblast cell lines when compared to control fibroblasts. **-p<0.01 ****-p<0.0001.

FIG. 12 shows concentration analyses of different fibroblast cell lines based on metabolomic data, which indicate highly elevated levels of the metabolite “Opthalmic acid” in all ten diseased fibroblast cell lines when compared to control fibroblasts. *-p<0.05; **-p<0.01; * p<0.001 ****-p<0.0001.

FIG. 13 shows concentration analyses of different fibroblast cell lines based on metabolomic data, which indicate highly elevated levels of the metabolite “S-adenosyl methionine” in all ten diseased fibroblast cell lines when compared to control fibroblasts. *-p<0.01; ***-p<0.001 ****-p<0.0001.

FIG. 14 shows concentration analyses of different fibroblast cell lines based on metabolomic data, which indicate highly elevated levels of the metabolite “Creatine” in eight out of the ten diseased fibroblast cell lines when compared to control fibroblasts. *-p<0.05; **-p<0.01; ***-p<0.001 ****-p<0.0001.

FIG. 15 is a biochemical pathway analysis that reveals a prominent role of putrescine and associated polyamine metabolites in all mitochondrial diseased fibroblasts.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Mitochondrial and metabolic disorders arise as a result of organ dysfunction and energy failure in tissues requiring energy to function. There is an urgent need to better understand the pathways and metabolite regulation in the context of mitochondrial and metabolic disorders. In particular, there is significant interest in studying these disorders because they may shed light into what happens in rare mitochondrial disorders in young children and common metabolic disorders arising as a result of aging. Moreover, human mitochondrial and metabolic disorders help elucidate the root cause of energy dysfunction in rare and common age-associated disorders of the brain, muscle, heart, kidney, immune system and many tissues that constitute the human body.

However, diagnosis of mitochondrial and metabolic disorders are extremely difficult due to clinical variance. As such, there is a need for specific and sensitive methods for the assessment of mitochondrial and metabolic disorders. Numerous embodiments of the present disclosure address the aforementioned need.

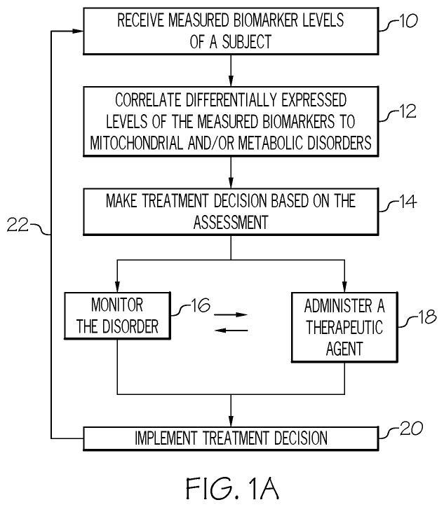

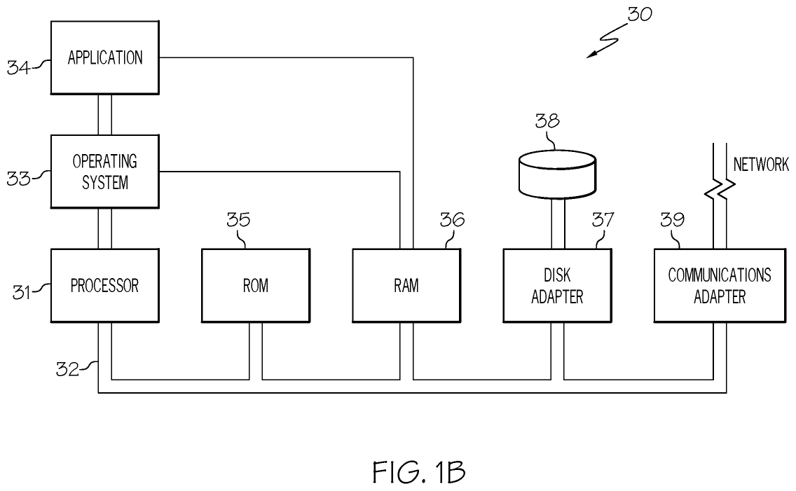

In some embodiments, the present disclosure pertains to methods of assessing one or more mitochondrial or metabolic disorders in a subject. In some embodiments illustrated in FIG. 1, the methods of the present disclosure include: receiving one or more measured biomarker levels of the subject (step 10); and correlating differentially expressed levels of the one or more measured biomarkers to one or more mitochondrial or metabolic disorders in the subject (step 12). In some embodiments, the methods of the present disclosure also include a step of making a treatment decision based on the assessment (step 14). In some embodiments, the treatment decision includes monitoring the course of the mitochondrial or metabolic disorder (step 16) and/or administering a therapeutic agent to the subject (step 18). In some embodiments, the methods of the present disclosure also include a step of implementing the treatment decision (step 20). In some embodiments, the assessment may be repeated after implementing the treatment decision (step 22).

Additional embodiments of the present disclosure pertain to a computing device for assessing one or more mitochondrial or metabolic disorders in a subject. In some embodiments, the computing device includes one or more computer readable storage mediums having a program code embodied therewith. In some embodiments, the program code includes programming instructions for: receiving one or more measured biomarker levels of the subject; and correlating differentially expressed levels of the one or more measured biomarkers to one or more mitochondrial or metabolic disorders in the subject. In some embodiments, the program code further includes programming instructions for diagnosing the subject with one or more mitochondrial or metabolic disorders based on the correlation. In some embodiments, the program code further includes programming instructions for assessing the severity of one or more mitochondrial or metabolic disorders in the subject based on the correlation.

In some embodiments, the program code further includes programming instructions for making a treatment decision based on the assessment. In some embodiments, the treatment decision includes monitoring the course of one or more mitochondrial or metabolic disorders, administering a therapeutic agent to the subject, or combinations thereof. In some embodiments, the program code further includes programming instructions for repeating the assessment after implementing the treatment decision.

As set forth in more detail herein, the methods and computing devices of the present disclosure can have numerous embodiments.

### Mitochondrial and Metabolic Disorders

The methods and computing devices of the present disclosure may be utilized to assess various mitochondrial or metabolic disorders. For instance, in some embodiments, the assessed mitochondrial or metabolic disorders include, without limitation, one or more mitochondrial diseases; metabolic diseases; obesity; cancer; neurodegenerative diseases; age-related neurodegenerative diseases; pediatric neurodegenerative diseases; immune disorders; diabetes; host-parasite infections; cardiovascular disease; Leigh syndrome (LS); Mitochondrial Encephalopathy, Lactic acidosis, Stroke-like episodes (MELAS); Kearns-Sayre Syndrome (KSS); Pearson Syndrome (PS); exercise intolerance syndrome; Alzheimer's disease; Parkinson's disease; multiple organ dysfunction syndrome; sepsis; fatty liver disease; kidney disease; hirsutism; African sleeping sickness; Synder Robinson syndrome; osteoporosis; Huntington's disease; multiple sclerosis; periodontitis; or combinations thereof. In some embodiments, the one or more mitochondrial or metabolic disorders includes LS. In some embodiments, the one or more mitochondrial or metabolic disorders includes MELAS. In some embodiments, the one or more mitochondrial or metabolic disorders includes KSS. In some embodiments, the one or more mitochondrial or metabolic disorders includes PS. In some embodiments, the one or more mitochondrial or metabolic disorders include one or more neurodegenerative disorders, such as Alzheimer's disease or Parkinson's disease.

### Biomarker Levels

The methods and computing devices of the present disclosure may correlate differentially expressed levels of various measured biomarkers to mitochondrial or metabolic disorders. For instance, in some embodiments, the measured biomarker levels include at least one of putrescine, N-acetyl putrescine, N-acetyl ornithine, N-acetyl spermidine, N-acetyl arginine, gamma amino butyric acid (GABA), opthalmic acid, S-adenosyl methionine, creatine, or combinations thereof. In some embodiments, the measured biomarker levels include a single measured biomarker level.

In some embodiments, the measured biomarker levels include a plurality of measured biomarker levels. In some embodiments, the plurality of measured biomarker levels includes at least two of the aforementioned measured biomarker levels. In some embodiments, the plurality of measured biomarker levels includes at least three of the aforementioned measured biomarker levels. In some embodiments, the plurality of measured biomarker levels includes at least four of the aforementioned measured biomarker levels. In some embodiments, the plurality of measured biomarker levels includes at least five of the aforementioned measured biomarker levels. In some embodiments, the plurality of measured biomarker levels includes at least six of the aforementioned measured biomarker levels. In some embodiments, the plurality of measured biomarker levels includes at least seven of the aforementioned measured biomarker levels. In some embodiments, the plurality of measured biomarker levels includes at least eight of the aforementioned measured biomarker levels. In some embodiments, the plurality of measured biomarker levels includes putrescine, N-acetyl putrescine, N-acetyl ornithine, N-acetyl spermidine, N-acetyl arginine, gamma amino butyric acid (GABA), opthalmic acid, S-adenosyl methionine, and creatine.

In some embodiments, the differentially expressed levels of the one or more biomarkers represent elevated levels of the one or more biomarkers, depressed levels of the one or more biomarkers, or combinations thereof. In some embodiments, the differentially expressed levels of the one or more biomarkers represent elevated levels of the one or more biomarkers. In some embodiments, the differentially expressed levels of the one or more biomarkers represent depressed levels of the one or more biomarkers.

In some embodiments, the methods of the present disclosure also include a step of measuring the one or more biomarker levels from a tissue sample, body fluid or blood sample obtained from a subject. In some embodiments, the methods of the present disclosure also include a step of obtaining a tissue sample, body fluid, or blood sample from the subject.

### Subjects

The methods and computing devices of the present disclosure may be utilized to assess mitochondrial or metabolic disorders in various subjects. For instance, in some embodiments, the subject is a human being. In some embodiments, the subject is vulnerable to having one or more mitochondrial or metabolic disorders. In some embodiments, the subject is suspected of having one or more mitochondrial or metabolic disorders. In some embodiments, the subject is suffering from one or more mitochondrial or metabolic disorders. In some embodiments, the subject has received treatment or is receiving treatment for one or more mitochondrial or metabolic disorders.

### Correlation of Biomarkers to Mitochondrial and Metabolic Disorders

The methods and computing devices of the present disclosure may utilize various methods and programming instructions to correlate differentially expressed levels of measured biomarkers to mitochondrial or metabolic disorders. For instance, in some embodiments, the correlating includes diagnosing the subject with one or more mitochondrial or metabolic disorders. In some embodiments, the correlating includes assessing the severity of one or more mitochondrial or metabolic disorders in the subject. In some embodiments, the correlating includes the dysfunction in one or more organs associated with one or more mitochondrial or metabolic disorders in the subject.

In some embodiments, the correlating occurs manually. In some embodiments, the correlating occurs automatically through the utilization of an algorithm. In some embodiments, the program code of a computing device of the present disclosure further includes the algorithm for the correlation. In some embodiments, the methods and computing devices of the present disclosure also include a step of or programing instructions for feeding one or more measured biomarker levels into the algorithm.

In some embodiments, the algorithm includes a machine-learning algorithm. In some embodiments, the machine-learning algorithm is trained to assess one or more mitochondrial or metabolic disorders in the subject based on one or more measured biomarker levels. In some embodiments, the machine-learning algorithm includes, without limitation, Li-regularized logistic regression algorithms, supervised learning algorithms, nearest neighbor algorithms, naïve-Bayes algorithms, decision tree algorithms, linear regression algorithms, support vector machines, neural networks, convolutional neural networks, ensembles, and combinations thereof.

Machine-learning algorithms may be trained to assess one or more mitochondrial or metabolic disorders in various manners. For instance, in some embodiments, the training includes: (1) feeding a first set of measured biomarker levels correlated to one or more mitochondrial or metabolic disorders into a machine-learning algorithm; (2) feeding a second set of measured biomarker levels correlated to the same mitochondrial or metabolic disorders into a machine-learning algorithm; and (3) training the machine-learning algorithm to correlate the measured biomarker levels to one or more mitochondrial or metabolic disorders by comparing the first set of measured biomarker levels with the second set of measured biomarker levels. In some embodiments, the first set of measured biomarker levels corresponds to the training data and the second set of measured biomarker levels corresponds to validation data. In this manner, the first set of measured biomarker levels is utilized for training, which is validated based on the second set of measured biomarker levels. In some embodiments, training of a machine-learning algorithm includes the adjustment of weights or parameters within the machine-learning algorithm so as to differentiate between the first and second set of biomarker levels.

### Treatment Decisions

In some embodiments, the methods and computing devices of the present disclosure also include a step of or programming instructions for making a treatment decision based on the assessment. In some embodiments, the treatment decision includes monitoring the course of one or more mitochondrial or metabolic disorders, administering a therapeutic agent to the subject, or combinations thereof. In some embodiments, the treatment decision includes monitoring the course of one or more mitochondrial or metabolic disorders. In some embodiments, the treatment decision includes administering a therapeutic agent to the subject.

In some embodiments, the methods and computing devices of the present disclosure also include a step of or programming instructions for implementing a treatment decision. In some embodiments, the method is repeated after implementing the treatment decision.

### Computing Devices

The computing devices of the present disclosure can have various architectures. For instance, embodiments of the present disclosure as discussed herein may be implemented using a computing device 30 illustrated in FIG. 1B. Computing device 30 represents a hardware environment for practicing various embodiments of the present disclosure.

Computing device 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for assessing one or more mitochondrial or metabolic disorders in a subject, such as in connection with FIGS. 1A, 2A-2C, 3A-3B, 4A-4B, 5A-5C, and 6-15.

Referring again to FIG. 1B, read-only memory (“ROM”) 35 is connected to system bus 32 and includes a basic input/output system (“BIOS”) that controls certain basic functions of computing device 30. Random access memory (“RAM”) 36 and disk adapter 37 are also connected to system bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for assessing one or more mitochondrial or metabolic disorders in a subject, such as in connection with FIGS. 1A, 2A-2C, 3A-3B, 4A-4B, 5A-5C, and 6-15, may reside in disk unit 38 or in application 34.

Computing device 30 may further include a communications adapter 39 connected to bus 32. Communications adapter 39 interconnects bus 32 with an outside network (e.g., wide area network) to communicate with other devices.

Aspects of the present disclosure are described herein with reference to flowchart illustrations and/or block diagrams of methods, apparatus (systems), and computing devices according to embodiments of the disclosure. It will be understood that computer-readable program instructions can implement each block of the flowchart illustrations and/or block diagrams, and combinations of blocks in the flowchart illustrations and/or block diagrams.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other devices to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Identification of a Panel of Nine Biomarkers Associated with Disease Severity Associated with Mitochondrial and Metabolic Dysfunction

This Example illustrates the identification of a panel of nine biomarkers associated with polyamine dyshomeostasis as correlating with disease severity associated with mitochondrial and metabolic dysfunction. In particular, Applicant performed high-throughput targeted metabolomic analyses of over 6,000 metabolites in a diverse group of human fibroblasts obtained from healthy and diseased patients diagnosed with mitochondrial and metabolic dysfunction. Although Applicant first started with rare single mitochondrial DNA mutation in a pediatric neurodegenerative disease such as Leigh syndrome (LS), Applicant's study expanded to include multiple diseased fibroblast cell lines with other mitochondrial DNA mutations and mitochondrial DNA deletions found in common age related disorders.

In this Example, Applicant has analyzed five metabolic disorders, such as Leigh syndrome (LS); Mitochondrial Encephalopathy, Lactic acidosis, Stroke-like episodes (MELAS); Kearns-Sayre Syndrome (KSS); Pearson Syndrome (PS); and exercise intolerance syndrome. The results from this Example identify major polyamine dyshomeostasis in the multiple diseased cell lines and shows that many metabolites associated with polyamine pathway are highly elevated in the diseased cell lines. This finding is significant because it identifies a panel of novel diagnostic biomarkers for mitochondrial and metabolic diseases that have inherent mitochondrial dysfunction.

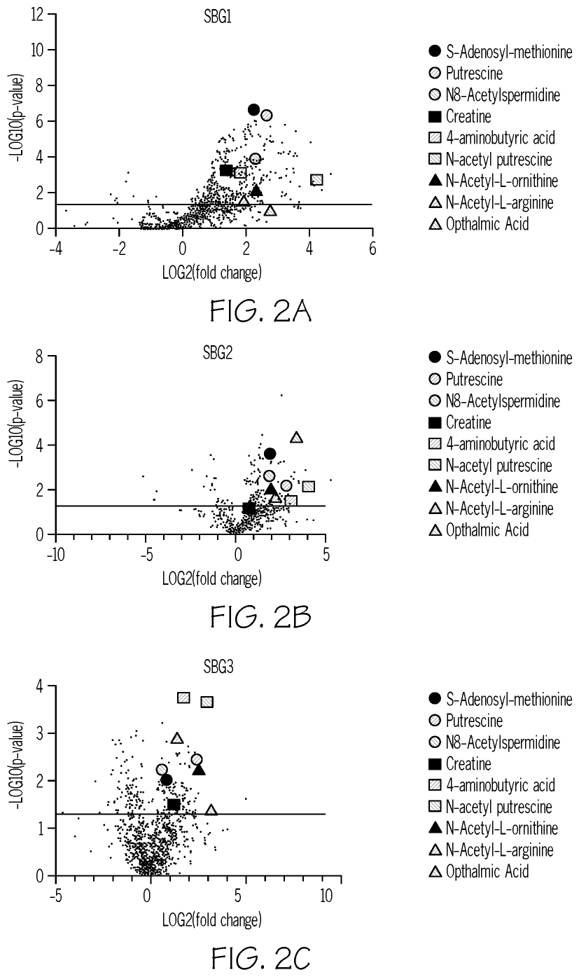

FIGS. 2A-2C show volcano plots obtained from metabolomics analysis of SBG1 (FIG. 2A), SBG2 (FIG. 2B), and SBG3 (FIG. 2C) diseased fibroblast cell lines. These cell lines carry point mutations in the MTATP6 subunits (SBG1, SGB2: m.8993T>G, SBG3: m.9185T>C) of complex V (ATP synthase) of the electron transport chain. These mutations have been implicated in Leigh Syndrome (LS), a classic mitochondrial disorder that affects mental and motor activity. Key metabolites with high p-values and fold change that are commonly highly elevated in multiple cell lines are highlighted. Positive fold change values indicate metabolites with higher concentrations in diseased fibroblasts than in controls.

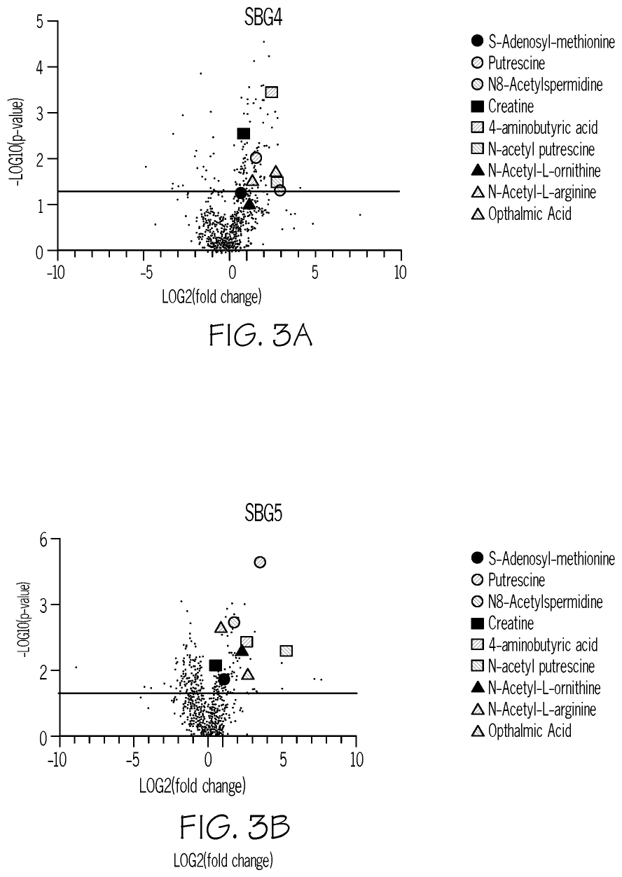

FIGS. 3A-3B show volcano plots obtained from metabolomics analysis of SBG4 (FIG. 3A) and SBG5 (FIG. 3B) diseased fibroblast cell lines. These cell lines carry point mutations that have mtDNA mutations affecting the MTND3 (SBG4: m.10158T>C) and MTND5 (SBG5: m.12706T>C) subunits of complex I of the electron transport chain. These mutations have been implicated in Leigh Syndrome (LS), a classic mitochondrial disorder that affects mental and motor activity. Key metabolites with high p-values and fold change that are commonly highly elevated in multiple cell lines are highlighted. Positive fold change values indicate metabolites with higher concentrations in diseased fibroblasts than in controls.

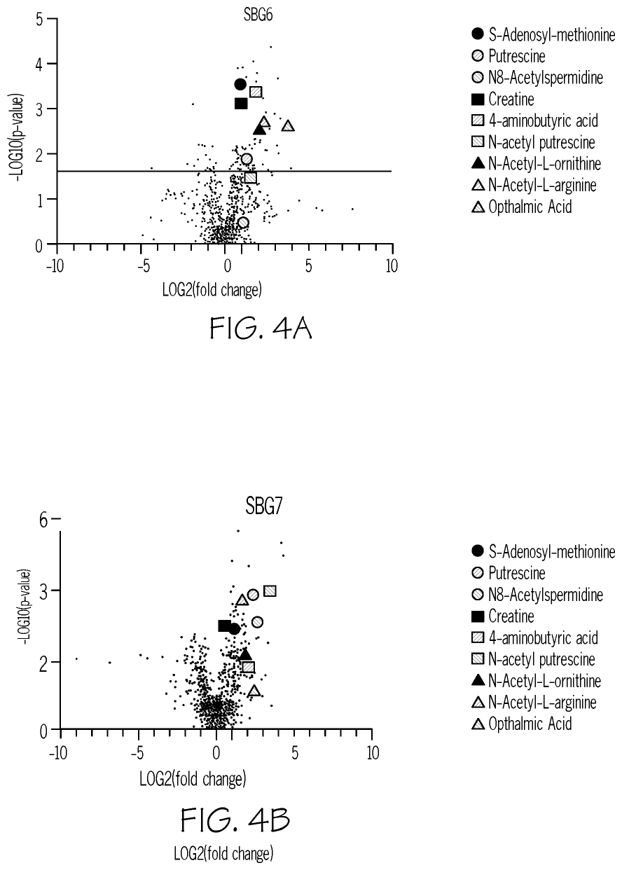

FIGS. 4A-4B show volcano plots obtained from metabolomics analysis of SBG6 (FIG. 4A) and SBG7 (FIG. 4B) diseased fibroblast cell lines. These cell lines have tRNA mutations (SBG6: m.3243A>G; SBG7: m.14739G>A mutation). These mutations have been implicated in MELAS (Mitochondrial Encephalopathy, Lactic acidosis, Stroke-like episodes), a condition that affects many of the body's systems, particularly the brain and the muscles with early symptoms in patients including muscle weakness and pain, recurrent headaches, loss of appetite, and seizures. Key metabolites with high p-values and fold change that are commonly highly elevated in multiple cell lines are highlighted. Positive fold change values indicate metabolites with higher concentrations in diseased fibroblasts than in controls.

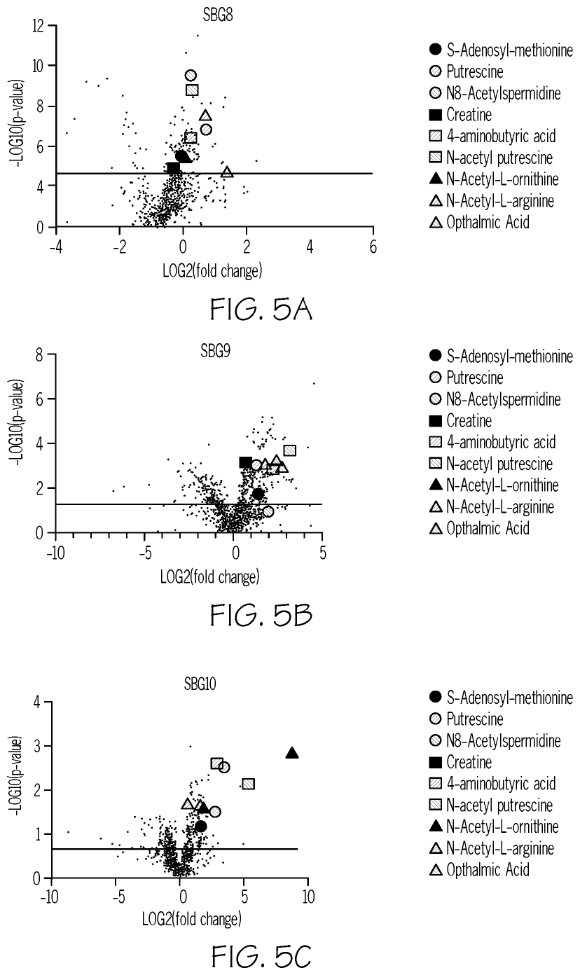

FIGS. 5A-5C show volcano plots obtained from metabolomics analysis of SBG8 (FIG. 5A), SBG9 (FIG. 5B), and SBG10 (FIG. 5C) diseased fibroblast cell lines. These cell lines have specific deletions in the mitochondrial genome (SBG8: 10676A14868; SBG9: 7342A9916; and SBG10: 10167A15568). These deletions have been implicated in KSS (Kearns sears syndrome) and PS (Pearson syndrome).

KSS is a condition that affects many parts of the body with patients exhibiting progressive external ophthalmoplegia, ptosis, pigmentary retinopathy, cardiac conduction defects, ataxia, or abnormally high levels of protein in the fluid that surrounds and protects the brain and spinal cord (the cerebrospinal fluid or CSF). PS usually begins in infancy with half of the children dying in infancy or early childhood due to severe lactic acidosis or liver failure, with those who survive developing signs and symptoms related to KSS. Key metabolites with high p-values and fold change that are commonly highly elevated in multiple cell lines are highlighted. Positive fold change values indicate metabolites with higher concentrations in diseased fibroblasts than in controls.

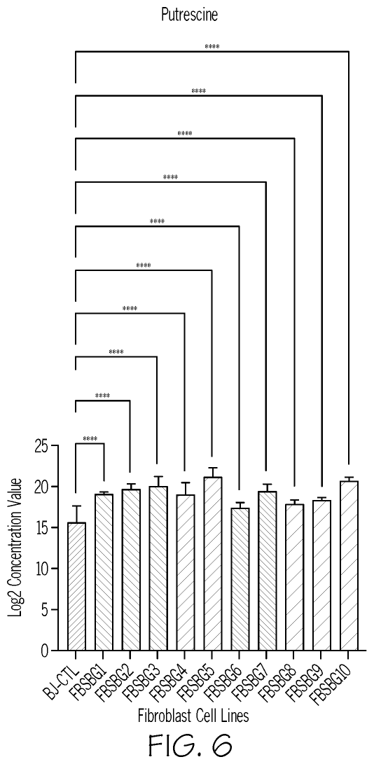

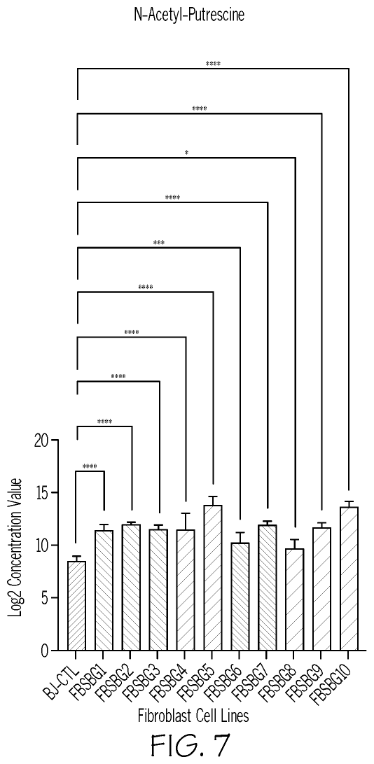

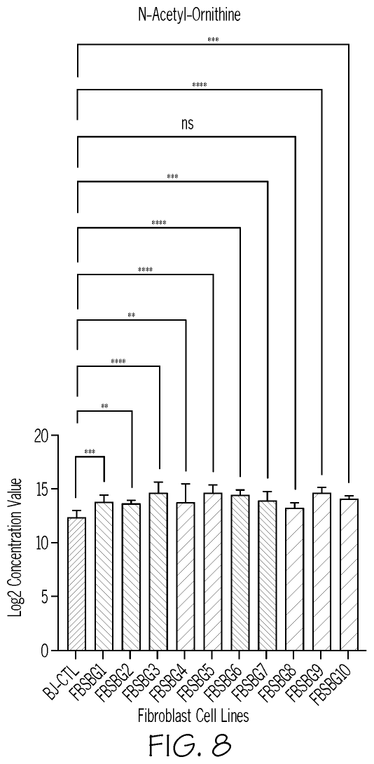

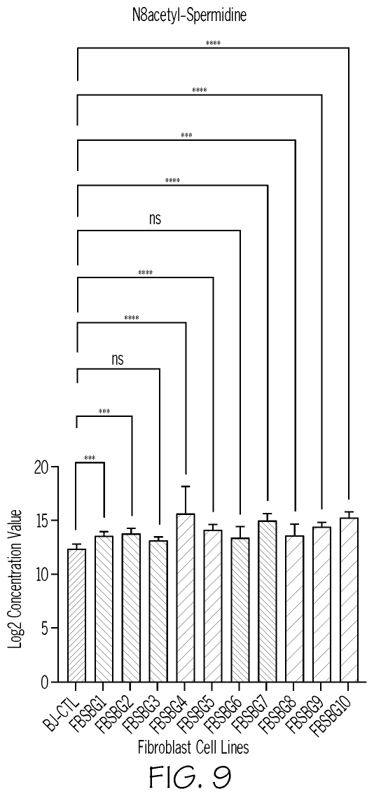

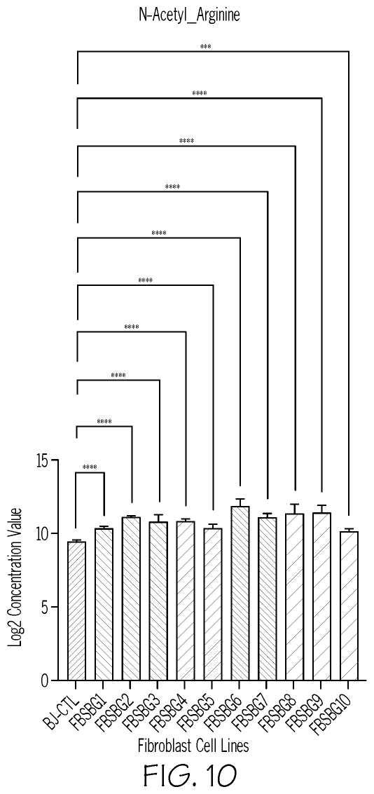

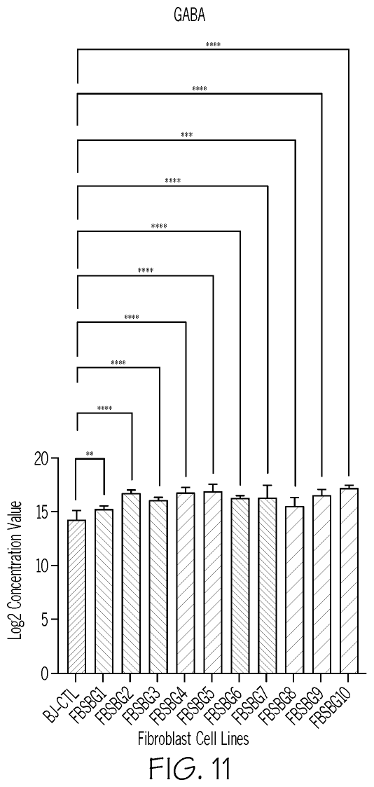

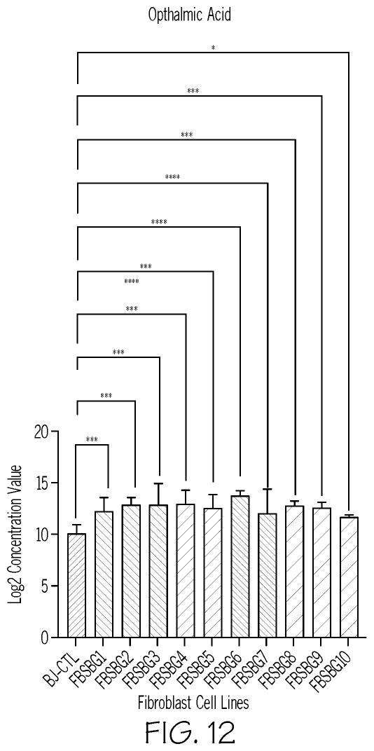

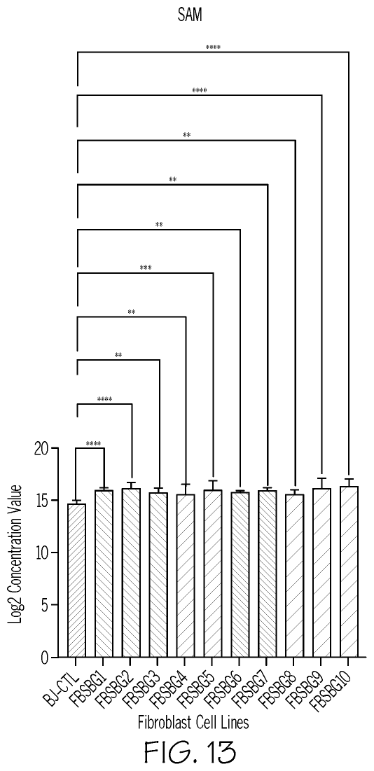

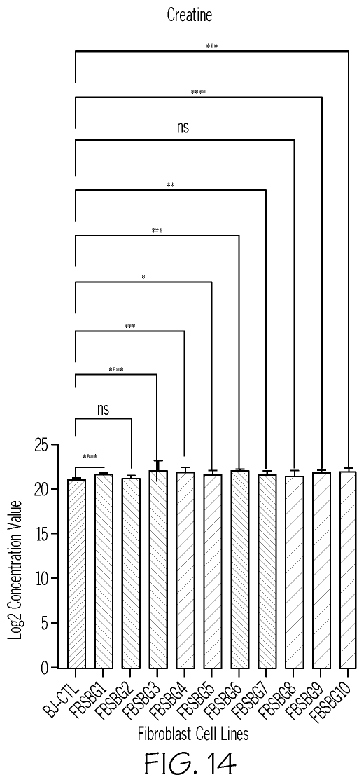

FIGS. 6-14 show metabolite concentration analyses based on metabolomic data in all ten diseased fibroblast cell lines when compared to control fibroblasts. Overall, the results indicate highly elevated levels of the metabolites putrescine (FIG. 6), N-acetyl putrescine (FIG. 7), N-acetyl ornithine (FIG. 8), N-acetyl spermidine (FIG. 9), N-acetyl Arginine (FIG. 10), Gamma Amino Butyric acid (FIG. 11, GABA), Opthalmic acid (FIG. 12), S-adenosyl methionine (FIG. 13, SAM), and Creatine (FIG. 14) in the cell lines.

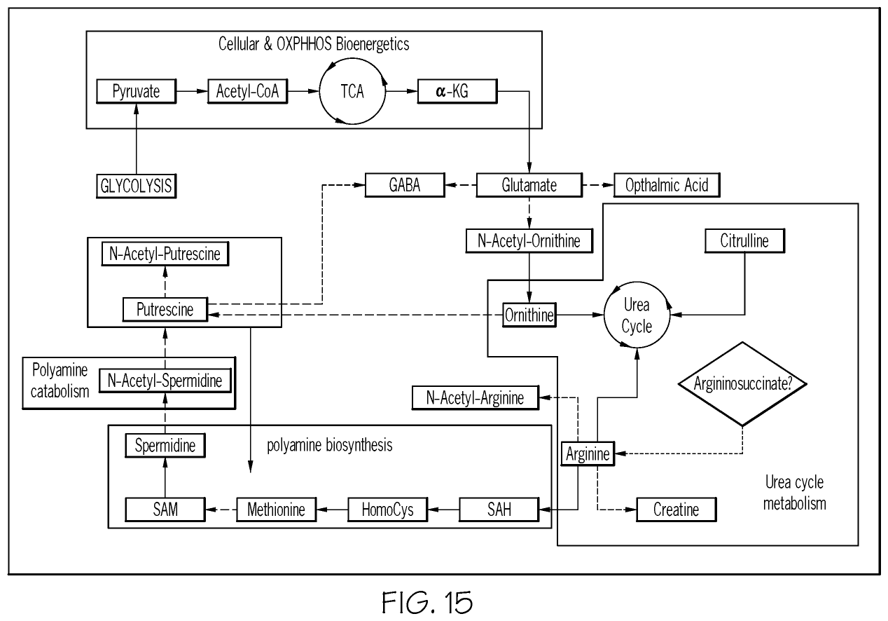

FIG. 15 illustrates a biochemical pathway analysis that reveals a prominent role of putrescine and associated polyamine metabolites in all mitochondrial diseased fibroblasts. The most affected panel of nine metabolites that is commonly upregulated in all the ten cell lines are putrescine, GABA, Ophthalmic acid, and acetylated polyamines (N-acetyl spermidine; N-acetyl ornithine; N-acetyl putrescine; N-acetyl arginine). S-adenosyl methinione, a metabolite associated with the methionine metabolism pathway is also highly elevated in all ten cell lines, indicating that the mitochondrial mutations and deletions alter the methinione metabolism pathway.

Accumulation of putrescine also triggers an alternate pathway to make GABA and Opthalmic acid, which are also elevated in all ten cell lines. Arginine is an intermediate in the urea cycle and is the precursor of creatine. Utilization of polyamines and GABA-based biomarkers could provide the necessary diagnostic information in aiding efficient and rapid diagnosis of mitochondrial disorders.

In sum, Applicant has identified a panel of nine biomarkers associated with polyamine dyshomeostasis as correlating with disease severity associated with mitochondrial and metabolic dysfunction. The panel of nine diagnostic novel biomarkers are highly elevated in multiple cell lines that exhibit a wide range of mitochondrial dysfunction, indicating their unique ability to be detected across mitochondrial and metabolic disorders. Use of these biomarkers has significant potential for efficient mitochondrial and metabolic disorder diagnosis in a wide range of diseases that exhibit mitochondrial and metabolic dysfunction.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of assessing one or more mitochondrial or metabolic disorders in a subject, said method comprising:
receiving one or more measured biomarker levels of the subject, wherein the one or more measured biomarker levels is selected from the group consisting of putrescine, N-acetyl putrescine, N-acetyl ornithine, N-acetyl spermidine, N-acetyl arginine, gamma amino butyric acid (GABA), opthalmic acid, S-adenosyl methionine, creatine, or combinations thereof; and
correlating differentially expressed levels of the one or more measured biomarkers to the one or more mitochondrial or metabolic disorders in the subject.

2. The method of claim 1, wherein the one or more mitochondrial or metabolic disorders is selected from the group consisting of mitochondrial diseases; metabolic diseases; obesity; cancer; neurodegenerative diseases; age-related neurodegenerative diseases; pediatric neurodegenerative diseases; immune disorders; diabetes; host-parasite infections; cardiovascular disease; Leigh syndrome (LS); Mitochondrial Encephalopathy, Lactic acidosis, Stroke-like episodes (MELAS); Kearns-Sayre Syndrome (KSS); Pearson Syndrome (PS); exercise intolerance syndrome; Alzheimer's disease; Parkinson's disease; multiple organ dysfunction syndrome; sepsis; fatty liver disease; kidney disease; hirsutism; African sleeping sickness; Synder Robinson syndrome; osteoporosis; Huntington's disease; multiple sclerosis; periodontitis; or combinations thereof.

3. The method of claim 1, wherein the one or more measured biomarker levels comprises putrescine, N-acetyl putrescine, N-acetyl ornithine, N-acetyl spermidine, N-acetyl arginine, gamma amino butyric acid (GABA), opthalmic acid, S-adenosyl methionine, and creatine.

4. The method of claim 1, wherein the differentially expressed levels of the one or more biomarkers represent elevated levels of the one or more biomarkers, depressed levels of the one or more biomarkers, or combinations thereof.

5. The method of claim 1, wherein the differentially expressed levels of the one or more biomarkers represent elevated levels of the one or more biomarkers.

6. The method of claim 1, wherein the subject is a human being.

7. The method of claim 1, wherein the correlating comprises diagnosing the subject with the one or more mitochondrial or metabolic disorders.

8. The method of claim 1, wherein the correlating comprises assessing the severity of the one or more mitochondrial or metabolic disorders in the subject.

9. The method of claim 1, wherein the correlating occurs automatically through the utilization of an algorithm.

10. The method of claim 9, wherein the algorithm comprises a machine-learning algorithm, wherein the machine-learning algorithm is trained to assess one or more mitochondrial or metabolic disorders in the subject based on the one or more measured biomarker levels.

11. The method of claim 1, further comprising a step of making a treatment decision based on the assessment.

12. The method of claim 11, wherein the treatment decision comprises monitoring the course of the one or more mitochondrial or metabolic disorders, administering a therapeutic agent to the subject, or combinations thereof.

13. The method of claim 11, wherein the treatment decision comprises administering a therapeutic agent to the subject.

14. The method of claim 11, further comprising a step of implementing the treatment decision.

15. A computing device for assessing one or more mitochondrial or metabolic disorders in a subject, wherein the computing device comprises one or more computer readable storage mediums having a program code embodied therewith, wherein the program code comprises programming instructions for:
receiving one or more measured biomarker levels of the subject, wherein the one or more measured biomarker levels is selected from the group consisting of putrescine, N-acetyl putrescine, N-acetyl ornithine, N-acetyl spermidine, N-acetyl arginine, gamma amino butyric acid (GABA), opthalmic acid, S-adenosyl methionine, creatine, or combinations thereof; and
correlating differentially expressed levels of the one or more measured biomarkers to the one or more mitochondrial or metabolic disorders in the subject.

16. The computing device of claim 15, wherein the program code further comprises an algorithm for the correlating.

17. The computing device of claim 16, wherein the algorithm comprises a machine-learning algorithm, wherein the machine-learning algorithm is trained to assess one or more mitochondrial or metabolic disorders in the subject based on the one or more measured biomarker levels.

18. The computing device of claim 15, wherein the program code further comprises programming instructions for diagnosing the subject with the one or more mitochondrial or metabolic disorders based on the correlating.

19. The computing device of claim 15, wherein the program code further comprises programming instructions for assessing the severity of the one or more mitochondrial or metabolic disorders in the subject based on the correlating.

20. The computing device of claim 15, wherein the program code further comprises programming instructions for making a treatment decision based on the assessment.

21. The computing device of claim 20, wherein the treatment decision comprises monitoring the course of the one or more mitochondrial or metabolic disorders, administering a therapeutic agent to the subject, or combinations thereof.

22. The computing device of claim 20, wherein the treatment decision comprises administering a therapeutic agent to the subject.

23. The computing device of claim 20, wherein the one or more measured biomarker levels comprises putrescine, N-acetyl putrescine, N-acetyl ornithine, N-acetyl spermidine, N-acetyl arginine, gamma amino butyric acid (GABA), opthalmic acid, S-adenosyl methionine, and creatine.
